(19) United States
(12) Patent Application Publication
(10) Pub. No.: US 2024/0410903 A1
(43) Pub. Date: Dec. 12, 2024

(54) METHODS FOR DETECTING FRACTURE-RELATED INFECTION (FRI)

(71) Applicants: THE TRUSTEES OF INDIANA UNIVERSITY, Bloomington, IN (US); PURDUE RESEARCH FOUNDATION, West Lafayette, IN (US); SAPIENZA UNIVERSITÀ DI ROMA, Rome (IT)

(72) Inventors: Roman M. NATOLI, Indianapolis, IN (US); Sarah MALEK, West Lafayette, IN (US); Federico MARINI, Rome (IT)

(21) Appl. No.: 18/698,129

(22) PCT Filed: Oct. 13, 2022
(86) PCT No.: PCT/US2022/046515

§ 371 Date: Apr. 3, 2024

Related U.S. Application Data
(60) Provisional application No. 63/256,394, filed on Oct. 15, 2021.

(51) Int. Cl. G01N 33/68

(52) U.S. Cl. CPC G01N 33/6893; G01N 33/6848; G01N 2800/26; G01N 2800/60

(57) ABSTRACT

Methods of detecting diagnostic indicators of a fracture-related infection (FRI) in a patient's biological sample are provided along with methods of treating the identified patients having an FRI.

### CROSS REFERENCE TO RELATED APPLICATIONS

This application claims priority to the following: U.S. Provisional Patent Application No. 63/256,394, filed on Oct. 15, 2021, the disclosure of which is expressly incorporated herein.

### BACKGROUND

Fracture-related infection (FRI) is a severe complication following bone injury. The incidence of fracture-related infection (FRI) varies widely depending on the injury, but it is commonly reported as 5-10%. The cost of FRIs exceeds $23,000 per infection, and there are more than 20,000 FRIs annually in the United States. Despite the significant socio-economic impact, the ability to diagnose FRIs remains a challenge. The infection work-up is largely based upon the history and physical exam, white blood cell (WBC) count, erythrocyte sedimentation rate (ESR), C-reactive protein (CRP), radiographs, and occasionally advanced imaging. Unfortunately, these diagnostic tools are of limited utility. Quantitative histology and tissue culture from intra-operative tissue samples can be useful tools to diagnose FRIs, but they are invasive, dependent on sample quality, and the results are not available until after the surgery has been performed.

Historically, only 2% of randomized controlled trials defined an “infection”. To address this, an expert panel was assembled in 2017 to better define a FRI. Unfortunately, like many of the prosthetic joint infection definitions, the FRI definition is composed of confirmatory and suggestive criteria. Confirmatory criteria include a sinus tract communicating with the implant, purulent drainage, phenotypically indistinguishable pathogens from two deep tissue cultures, or the presence of microorganisms on histopathologic examination. Suggestive criteria include clinical and radiologic signs, elevated ESR, WBC and/or CRP, and non-purulent wound drainage.

There is limited utility of the currently used common biomarkers, namely WBC, ESR, and CRP, for diagnosing FRI. Additionally, only a few studies have evaluated other biomarkers, such as the cytokine IL-6. A large systematic review of 8,284 articles looking at the diagnostic accuracy of these “classic” serum inflammatory markers determined they are insufficient. In that review, sensitivity, and specificity for CRP ranged from 60-100% and 34-86%, respectively.

Accordingly, a need exists for a method capable of accurately detecting a fracture-related infection including biomarkers with high specificity and sensitivity and an algorithm that assesses and determines the concentration of biomarkers accurately.

### SUMMARY

The disclosure applies the discovery of a combination of biomarkers that accurately identify the presence of a fracture-related infection (FRI). FRIs are generally observed in patients after a surgery to introduce, replace, or adjust an implant. In one example, an FRI may occur after broken bones are re-set and stabilized using medical grade implants (e.g., bone plate and screws); also referred to “fracture fixation”. Historically, it is difficult to discern a fracture-related infection from inflammation and discomfort associated with recovery from surgery. The recommended process of identifying an FRI include blood sample analysis, imaging, performing biopsies from two separate locations, tissue culture, and histology analysis. All of these tests and analysis are required just to confirm an FRI before treatment options are discussed. Preventative measures have been added in the form of surgery sanitation protocols and antimicrobial coatings on implants; however, with the continued rise in antibiotic resistant microbes, these practices are not efficient to address the burdensome undertaking just to identify an FRI.

In one aspect, the disclosure provides a method of detecting an FRI in a subject comprising analyzing a blood sample and quantifying the concentration of proteins. In some embodiments, the proteins diagnostic for an FRI are selected from C-reactive protein (CRP), interleukin 6 (IL-6), platelet-derived growth factor AB BB (PDGF-AB BB), and vascular endothelial growth factor A (VEGF-A), or a combination thereof. In some embodiments, the proteins diagnostic for an FRI include two or more proteins selected from CRP, IL-6, PDGF-AB BB, and VEGF-A.

In some embodiments, the proteins diagnostic for an FRI are selected from interleukin 6 (IL-6), platelet-derived growth factor AB BB (PDGF-AB BB), and vascular endothelial growth factor A (VEGF-A), optionally in combination with C-reactive protein (CRP) or other combination thereof. In some embodiments, the proteins diagnostic for an FRI include the proteins CRP, IL-6, PDGF-AB BB, and VEGF-A, optionally in combination with CRP.

In another aspect, the disclosure provides a method of detecting an FRI by obtaining a spectral profile and comparing the spectral peaks to a model/control to detect the presence of a spectral pattern associated with an FRI.

In one embodiment a method of treating an FRI is provided, once an FRI has been identified using one of the diagnostic methods disclosed herein. In one embodiment the treatment comprises using antimicrobial therapies known to the skilled practitioner, including for example the use of one or more antibiotics.

### BRIEF DESCRIPTION OF THE DRAWINGS

FIG. 1 is a graph showing the plasma concentration of C-reactive protein (CRP) differences between the confirmed infection group and control group.

FIG. 2 is a graph showing the plasma concentration of interleukin-6 (IL-6) differences between the confirmed infection group and control group.

FIG. 3 is a graph showing the plasma concentration of platelet-derived growth factor (PDGF)-AB BB differences between the confirmed infection group and control group.

FIG. 4 is a graph showing the plasma concentration of vascular endothelial growth factor (VEGF)-A differences between the confirmed infection group and control group.

FIG. 5 is a graph showing the Receiver Operator Characteristics (ROC) of CRP.

FIG. 6 is a graph showing the ROC of IL-6.

FIG. 7 is a graph showing the ROC of PDGF-AB BB.

FIG. 8 is a graph showing the ROC of VEGF-A.

FIG. 9 is a diagram showing the workflow of analytical steps for spectral data and ELISA-based measured values of plasma samples from FRI and control groups.

FIG. 10 is a graph showing an ROC curve for enzyme-linked immunosorbent assay (ELISA)-based biomarkers, wherein the ROC curve was calculated based on predictions from the reporting of the repeated (×50) double-cross-validation (rDCV) outer loops (both the ones corresponding to the individual DCV runs and their average (thick dark line)). X axis is 1-specificity (false positive rate), and Y axis is sensitivity (false negative rate).

FIG. 11 is a graph showing an ROC curve for Fourier-transform infrared (FTIR) spectral data, wherein the ROC curve was calculated based on predictions from the reporting of the repeated (×50) double-cross-validation (rDCV) outer loops (both the ones corresponding to the individual DCV runs and their average (thick dark line)). X axis is 1-specificity (false positive rate), and Y axis is sensitivity (false negative rate).

FIG. 12 is a graph showing an ROC curve when including both ELISA-based biomarkers and FTIR spectral data, wherein the ROC curve was calculated based on predictions from the reporting of the repeated (×50) double-cross-validation (rDCV) outer loops (both the ones corresponding to the individual DCV runs and their average (thick dark line)). X axis is 1-specificity (false positive rate), and Y axis is sensitivity (false negative rate).

FIG. 13 is a graph showing results of covariance selection-partial least squares-discriminant analysis (CovSel-PLS-DA) modeling on ELISA-based protein biomarker data, wherein double-cross-validated projections of the outer loop samples onto the only canonical variate of the classification model shows the difference in the values of the scores (bars indicate mean and whiskers the corresponding 95% confidence intervals) between FRI and control patients' samples.

FIG. 14 is a graph showing results of CovSel-PLS-DA modeling on ELISA-based protein biomarker data and the weights of the selected markers for the construction of that projection direction (canonical variate). FRI, fracture related infection group; Ctrl, control group; PDGF-AB/BB, platelet-derived growth factor AB/BB; CRP, C-reactive protein; MIG, monokine induced by gamma interferon.

FIG. 15 is a graph showing the results of CovSel-PLS-DA modeling on IR spectroscopic data and identifies the six variables (wavenumbers) selected as relevant by the CovSel algorithm (3288.7, 1648.6, 1624.3, 1592.9, 1188.2 and 610.6 cm-1; red vertical bars) over the mean IR spectrum of the samples.

FIG. 16 is a graph showing the results of CovSel-PLS-DA modeling on IR spectroscopic data, and the double-cross-validated projections of the outer loop samples onto the only canonical variate of the classification model showing the difference in the values of the scores (bars indicate mean and whiskers the corresponding 95% confidence intervals) between FRI and control patient samples.

FIG. 17 is a graph showing the results of CovSel-PLS-DA modeling on IR spectroscopic data, and the weights of the selected markers for the construction of that projection direction (canonical variate). FRI, fracture related infection group; Ctrl, control group.

FIG. 18 is a graph showing results of PLS-DA data fusion of selected ELISA-based biomarkers and FTIR spectral variables, and the double-cross-validated projections of the outer loop samples onto the only canonical variate of the classification model showing the difference in the values of the scores (bars indicate mean and whiskers indicate the corresponding 95% confidence intervals) between FRI and control patients' samples.

FIG. 19 is a graph showing results of PLS-DA data fusion of selected ELISA-based biomarkers and FTIR spectral variables and the weights of the selected markers for the construction of that projection direction (canonical variate). FRI, fracture related infection group; Ctrl, control group; CV, coefficient variable; PDGF-AB/BB, platelet-derived growth factor AB/BB; CRP, C-reactive protein; MIG, monokine induced by gamma interferon.

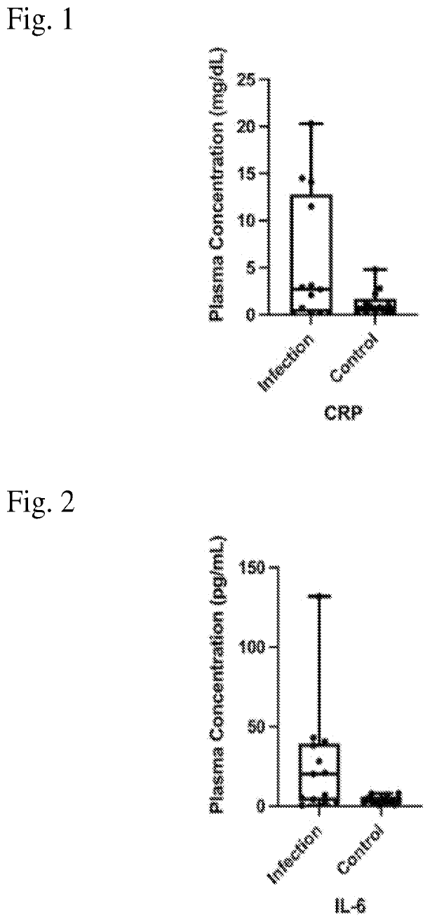

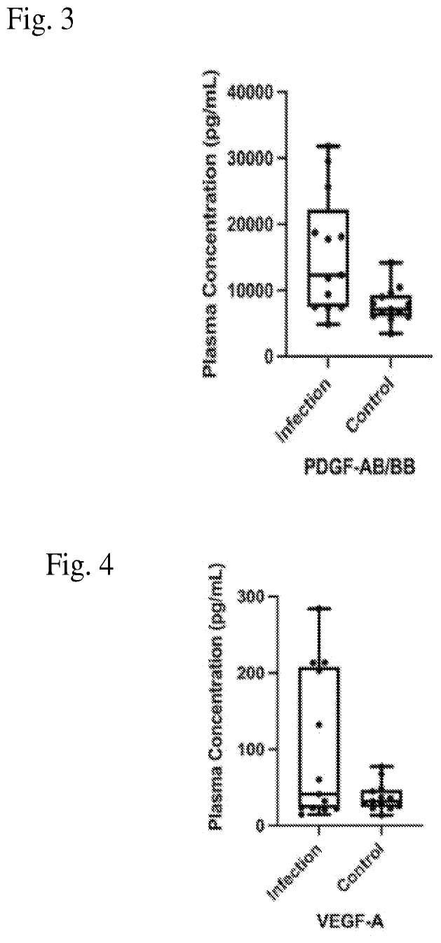

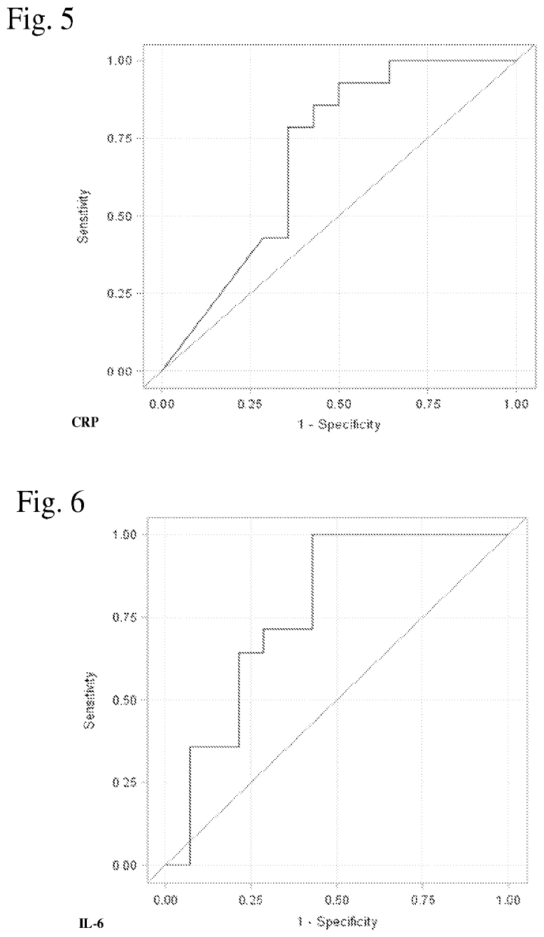

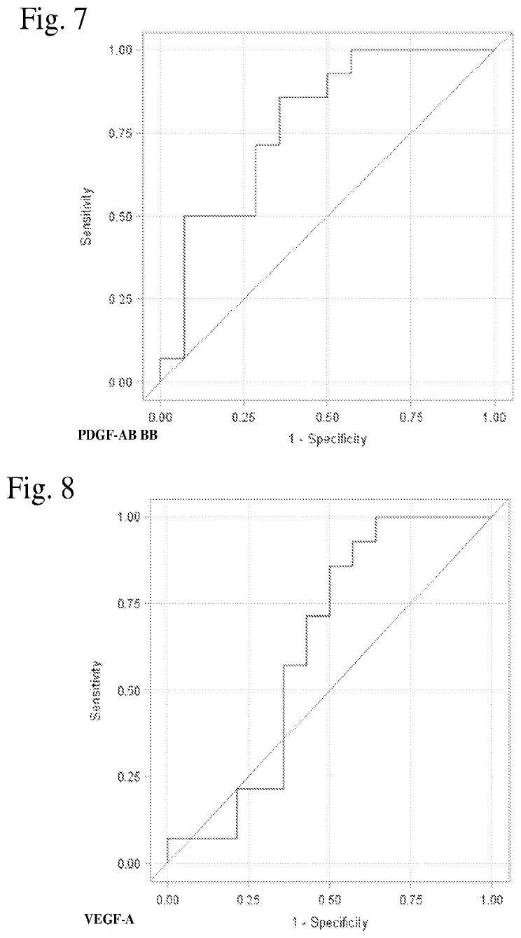

### DETAILED DESCRIPTION

### Abbreviations

FRI stands for fracture-related infection.
ROC stands for receiver operator characteristics.
AUC stands for area under the curve.
WBC stands for white blood cell.
CRP stands for C-reactive protein.
ESR stands for erythrocyte sedimentation rate.
IL-6 stands for interleukin 6.
PDGF-AB BB stands for platelet-derived growth factor AB BB.
VEGF-A stands for vascular endothelial growth factor A.
PJI stands for periprosthetic joint infection.
MIG stands for monokine induced by gamma interferon.

### Definitions

In describing and claiming the methods, the following terminology will be used in accordance with the definitions set forth below.

The term “effective” amount or a “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “cut point” defines a threshold concentration that indicates the likely presence of an FRI. In some aspects, the cut point is used to optimize the detection and concentration of the protein biomarkers, e.g., in analysis of an ELISA assay.

The term “subject” or “patient” means an animal including, but not limited to, humans, domesticated animals including horses, dogs, cats, cattle, and the like, rodents, reptiles, and amphibians receiving a therapeutic treatment either with or without physician oversight. In some respects, an animal may be referred to as a subject or a patient. In some embodiments, the subject is a post-operative patient having undergone surgery to introduce, replace, or adjust an implant.

The term “fracture” a used herein refers to any injury or breakage of bones, and includes damage to bones ranging from small hairline fractures to traumatic bone breaks.

The term “fracture-related infection (FRI)” encompasses all infections which occur in the presence of a fracture or the introduction, replacement, or adjustment of an implant. This includes early infection around fracture implants, infected non-unions, hematogenous infections arising after fracture healing and infections in fractures with no internal fixation as well as infections associated with implants.

### EMBODIMENTS

Embodiments of the disclosure include a method of detecting an FRI and a system for detecting an FRI using antibodies, spectroscopic profile(s), or both as well as methods for treating patients who have been identified as having an FRI.

The method includes the detection of FRIs occurring on any part of the skeleton. The fracture associated with the FRI may include one or more fractures of the same bone or cartilage. Alternatively, there may be more than one fracture on more than one bone and/or cartilage.

### Protein Biomarkers

To streamline and reduce the number of tests required to diagnose a FRI, the disclosure provides a method of detecting and analyzing protein biomarkers with specificity for FRI. In this way, it is envisioned that the financial cost and time to diagnose an FRI will be significantly reduced.

In one illustrative aspect, a method is provided for detecting the presence of an FRI. The method includes detecting and analyzing protein biomarkers in a sample obtained from a subject. The protein biomarkers may be produced during or cause an inflammatory response. The protein biomarkers may be obtained from any biological sample recovered from the patient including urine or blood. In one embodiment the biological sample is a whole blood, serum or plasma sample.

The subject may or may not be suspected of having an FRI. The subject may have a permanent or temporary implant. The subject may have undergone fracture fixation surgery to repair at least one fracture on a bone of the subject. The subject may have had surgery to repair multiple fractures on one or more bones. The fracture may be open or closed.

The subject's bone may be any bone in the human skeleton. The bone may be homologous or heterologous.

In one aspect, the method includes the detection of a biomarker above or below a certain concentration threshold. The concentration threshold is relative to a matching subject that does not have an FRI. In some embodiments, a biomarker detected in a subject sample at a concentration higher than that of a control sample would indicate that the subject has FRI. The method may include the detection of at least one, at least two, at least three, or at least four protein biomarkers. Each biomarker may be detected at a concentration threshold higher or lower than a control concentration. For example, where three biomarkers are detected and two are detected at a lower concentration thresholds than the control concentrations, the subject's probability of not having an FRI is more likely than if only one of the three biomarkers had a concentration detected at or below a concentration threshold. Having three of the diagnostic proteins of the present disclosure detected below the respective threshold values reliably predicts no FRI better than detecting only two below their respective threshold values. When all four are below the respective threshold, specificity is 100%.

In some aspects where at least four of the diagnostic proteins of the present disclosure are selected to be detected in a subject's sample, the detection of at least one, at least two, at least three, or at least four of the protein biomarkers at a concentration at or higher than each biomarker's concentration threshold would indicate the presence of FRI. When the method is designed to detect one biomarker above the thresholds, the sensitivity of diagnosis is about 85% for the presence of FRI.

The method may include the detection of a biomarker in a subject's sample and analyzing and determining the concentration of that biomarker. In some embodiments, the biomarker is selected from PDGF-AB BB, VEGF-A, IL-6, CRP, MIG, or a combination thereof. The method may include the detection of PDGF-AB BB and at least one other biomarker selected from VEGF-A, IL-6, MIG, or CRP. In other aspects, the method may include the detection of PDGF-AB BB, VEGF-A, IL-6, MIG and CRP. The method may include the detection of PDGF-AB BB, CRP, and MIG. Additionally, the method includes analyzing and determining the concentration of the biomarker. The concentration is then referenced to the cut-point, to determine the probability of FRI.

In one embodiment, the method includes detection of PDGF-AB BB from a sample derived from a subject. The detection of PDGF-AB BB at a concentration at or above about 12,000 pg/mL, about 11,500 pg/mL, about 11,000 pg/mL, about 10,500 pg/ml, or about 10,000 pg/mL indicates the presence of a FRI. In another aspect the cut-point is at or below about 10,550 pg/mL, about 10,500 pg/mL, about 10,450 pg/mL, about 10,400, or about 10, 350 pg/mL. The cut-point may be about 10, 445 pg/mL, about 10, 444 pg/mL, about 10,443 pg/mL, about 10,442 pg/mL, about 10,441 pg/mL, or about 10,440 pg/mL.

The method may include detecting VEGF-A from a sample derived from a subject. The detection of VEGF-A at a concentration at or above about 80 pg/mL, 79.5 pg/mL, 79 pg/mL, 78.5 pg/mL, 78 pg/mL, 77.5 pg/mL, 77 pg/mL, 76.5 pg/mL, 76 pg/mL or 75.5 pg/mL indicates the presence of an FRI.

The method may include detecting IL-6 from a sample derived from a subject. The detection of IL-6 at a concentration at or below about 8.2 pg/mL, 8.1 pg/mL, 8.0 pg/mL, 7.9 pg/mL, 7.8 pg/mL, 7.7 pg/mL, 7.6 pg/mL, or 7.5 pg/mL indicates the presence of an FRI.

The method may include detecting CRP from a sample derived from a subject. The detection of CRP at a concentration at or below about 3.1 mg/dl, 3.0 mg/dL, 2.9 mg/dl, 2.8 mg/dL, 2.7 mg/dL, 2.6 mg/dL, or 2.5 mg/dL indicates the presence of an FRI.

The plasma or whole blood sample may be obtained from a subject up to six months after initial surgery to repair the fracture. In some instances, the sample may be obtained between about 1 day to about 6 months after fracture fixation surgery. In some instances, the sample is obtained 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 28 day, 32 days, two months, three months, four months, or five months after fracture fixation surgery. In one embodiment the sample is obtained at about 1 week, about 2 weeks, about 3 weeks, 4 weeks, about 5 weeks, or about 6 weeks after fracture fixation surgery. The sample may be fresh or frozen and thawed prior to analysis.

The protein biomarkers may be detected and analyzed using an antibody-based assay, such as an ELISA or any other detection method known to those skilled in the art. In accordance with one embodiment a kit for detecting a FRI is provided, wherein the kit comprises antibodies specific for each of PDGF-AB BB, VEGF-A, IL-6, and CRP. In one embodiment the kit further comprises an antibody specific for MIG. In one embodiment the antibodies are monoclonal antibodies. In one embodiment the antibodies are labeled with a detectable marker. In one embodiment the antibodies are covalently linked to a solid support.

### Spectral Biomarker

In accordance with one embodiment a profile of a patient's biological sample (e.g., a urine, serum or plasma sample) as determined by spectroscopic analysis can be used to detect the presence of an FRI. In one embodiment a method for obtaining a mid-infrared (MIR) spectroscopic profile of a plasma sample (or other biological fluid recovered from a patient) using Fourier-transform infrared spectroscopy (FTIR) is provided. The method comprises acquiring FTIR spectra based on measurements of plasma samples using a dried film technique. In this method the plasma is dried on a microplate that is then read by the machine (FTIR spectrometer) and the spectral pattern is displayed in form of a unique waveform. This waveform (i.e., spectrum) undergoes preprocessing before the analytical modeling is conducted. Preprocessing involves a variety of steps that reduces redundant information and noise from the spectra (e.g., scatter correction and derivative techniques). Multivariate analytical methods are needed for comparing spectra and development of predictive models. The predictive model algorithms based on spectra can identify features that are unique to the disease state (i.e., FRI) compared to controls. The MIR spectroscopic profile may be referred to as a “spectral biomarker”, “biochemical fingerprint,” a “spectral fingerprint,” or simply a “fingerprint.”

In a further aspect, there is provided a system for obtaining a serum MIR spectroscopic profile using Fourier-transform infrared spectroscopy (FTIR). In one embodiment the system comprises one or more of a FTIR spectrometer, a preprocessing module, a normalization module, and a user interface. The FTIR spectrometer is used for obtaining FTIR spectra from the plasma samples. The preprocessing module may preprocess the FTIR spectra by differentiation and smoothing to enhance weak spectral features and to remove baseline variations, or other validated methods. In the development phase, the user interface utilizes the analytical methods for spectral analysis to develop the predictive model algorithms based on these spectra to identify FRI spectra from control healthy spectra. These developed predictive algorithms are then embodied in the form of a software that would read spectra from new plasma samples and classify them as FRI versus control based on their “spectral fingerprint”.

In an embodiment, the system further comprises a pattern recognition module for identifying, in the serum spectroscopic profile, spectroscopic features conveying diagnostic information of interest using pattern recognition models and a diagnostic module for diagnosing a fracture-related infection.

In a further aspect, there is provided a machine-readable medium containing sets of instructions, code sequences, configuration information, or other data, which, when executed, cause a processor to perform steps in a method for obtaining a serum mid-IR spectroscopic profile using FTIR. The method comprises acquiring FTIR spectra for dried plasma and preprocessing the FTIR spectra. The preprocessed FTIR spectra are normalized to a common intensity range, the normalization being performed in a spectral sub-region defined by strongest IR absorption for a protein to obtain the serum spectroscopic profile for providing a basis to diagnose an FRI.

For diagnostic analysis, a pattern recognition technique may be used to seek specific spectral ranges within which the spectra differed for normal specimens and those having an FRI. The pattern recognition model is optimized using the predictive algorithms disclosed and exemplified herein. Generally, the predictive algorithms were developed using spectral data obtained from FTIR spectroscopy performed on samples from confirmed FRI and control samples. The algorithms were designed to be implemented on spectrometers (portable or stationary) to detect the spectral data indicative of an FRI.

In one aspect, the pattern recognition model disclosed herein identified up to six wavenumbers of interest to differentiate between an FRI patient and a matching control patient. The method may include the detection of one or more predictive wavenumber variables (i.e., in this study 610.6, 1188.2, 1592.9, 1624.3, 1648.6 and 3288.7 cm⁻¹). In one aspect, the pattern recognition model may detect higher absorbance of 1624.3 and 1188.2 cm⁻¹ and lower absorbance at 610.6, 1592.9, 1648.6 and 3288.7 cm⁻¹ in an FRI patient than a non-FRI patient.

The model may incorporate the spectroscopic profile of a patient along with the biomarker analysis to increase specificity for detecting FRI.

In accordance with one embodiment a method of treating an FRI in a subject is provided wherein the method comprises:
determining if a subject's plasma sample exhibits a biomarker associated with FRI, to identifying a subject having an FRI, wherein the biomarker is
   i) a spectroscopic profile of a plasma sample associated with FRI or
   ii) an increased relative expression of one or more proteins selected from the group consisting of interleukin 6 (IL-6), platelet-derived growth factor AB BB (PDGF-AB BB), and vascular endothelial growth factor A (VEGF-A); and administering to said subject identified as having an FRI an anti-infection therapy, optionally wherein the anti-infection therapy is the administration of antibiotics.

In accordance with one embodiment a method of detecting an FRI is provided wherein a plasma sample is subjected to spectrometer analysis to generate a spectroscopic profile. The resulting profile is compared to a reference spectroscopic profile generated from a plasma sample from a healthy subject, and differences in the spectral peaks are assessed to determine the presence of peaks associated with an FRI. In one embodiment the detection of an FRI is associated with elevated expression levels of one or more proteins selected from the group consisting of the group consisting of C-reactive protein (CRP), interleukin 6 (IL-6), platelet-derived growth factor AB BB (PDGF-AB BB), and vascular endothelial growth factor A (VEGF-A).

In one embodiment a method of detecting an FRI in a subject is provided where the method comprises:
obtaining a test spectroscopic profile of a plasma sample obtained from the subject, and
comparing the test spectroscopic profile to a control spectroscopic profile of a plasma sample, wherein higher absorbance at about 1624-1625 cm⁻¹ and/or about 1188-1189 cm⁻¹ and lower absorbance at about 610-611 cm⁻¹, 1592-1593 cm⁻¹, 1648-1649 cm⁻¹, and/or 3288-3289 cm⁻¹ of the test spectroscopic profile when compared to the control spectroscopic profile indicates that the subject likely has an FRI, whereupon an FRI in the subject is detected.

In one embodiment a method of treating an FRI in a subject having FRI is provided, said method comprising:
identifying a subject having an FRI, as a subject having elevated levels of two, three or four proteins selected from the group consisting of C-reactive protein (CRP), interleukin 6 (IL-6), platelet-derived growth factor AB BB (PDGF-AB BB), and vascular endothelial growth factor A (VEGF-A), wherein the identification is determined by
   i) obtaining a reference expression level of proteins selected from the group consisting of C-reactive protein (CRP), interleukin 6 (IL-6), platelet-derived growth factor AB BB (PDGF-AB BB), and vascular endothelial growth factor A (VEGF-A);
   ii) determining an expression level of two, three or four proteins in a sample obtained from a test subject, wherein the two, three or four proteins correspond to those detected in step i), and identifying a subject having FRI as a test subject having an increase in the expression level of said three or more proteins in said patient's sample as compared to the reference expression level; and
administering to said patient having an FRI an anti-infection therapy, optionally wherein the anti-infection therapy is the administration of antibiotics. The method of detecting the expression level of three or more proteins in a sample obtained from a test subject, can be by any detection/quantification technique known to the skilled practitioner including for example using ELISA assays, spectroscopy, or mass spectrometry. In one embodiment the step of identifying a subject having an FRI comprises identification of a subject having elevated levels of all four proteins selected from the group consisting of C-reactive protein (CRP), interleukin 6 (IL-6), platelet-derived growth factor AB BB (PDGF-AB BB), and vascular endothelial growth factor A (VEGF-A),

In one embodiment a method of detecting and treating a fracture-related infection (FRI) in a subject is provided, wherein the method comprises:
obtaining a test spectroscopic profile of a plasma sample obtained from the subject, and
comparing the test spectroscopic profile to a control spectroscopic profile of a plasma sample, wherein higher absorbance at about 1624-1625 cm⁻¹ and/or about 1188-1189 cm⁻¹ and/or lower absorbance at about 610-611 cm⁻¹, 1592-1593 cm⁻¹, 1648-1649 cm⁻¹, and/or 3288-3289 cm⁻¹ of the test spectroscopic profile when compared to the control spectroscopic profile indicates that the subject likely has an FRI, and treating the subject for FRI, optionally with an antimicrobial agent. In one embodiment the serum sample is subjected to analysis by an FTIR spectrometer. In one embodiment the subject is determined to have an FRI, if the higher absorbance is at 1624.3 cm⁻¹ and/or 1188.2 cm⁻¹ and/or the lower absorbance is at 610.6 cm⁻¹, 1592.9 cm⁻¹, 1648.6 cm⁻¹, and/or 3288.7 cm⁻¹ or if the higher absorbance is at 1624.3 cm⁻¹ and 1188.2 cm⁻¹ and the lower absorbance is at 610.6 cm⁻¹, 1592.9 cm⁻¹, 1648.6 cm⁻¹, and 3288.7 cm⁻¹.

In one embodiment the method of determining if a subject has an FRI comprises: obtaining a test protein biomarker profile of a plasma sample obtained from the subject, and comparing the test protein biomarker profile to a control protein biomarker profile of a plasma sample from a healthy subject, wherein C-reactive protein (CRP) below about 2-3 mg/dL, interleukin 6 (IL-6) below about 7-8 pg/mL, platelet-derived growth factor-AB BB (PDGF-AB BB) below about 10,442-10,444 pg/mL, and/or vascular endothelial growth factor A (VEGF-A) below about 77-78 pg/mL indicates that the subject unlikely has an FRI. In one embodiment the protein biomarker profile is obtained using a detectably labeled antibody for CRP, a detectably labeled antibody for IL-6, a detectably labeled antibody for PDGF-AB BB, and/or a detectably labeled antibody for VEGF-A. In one embodiment the subject is determined unlikely to have an FRI, if the detected CRP is below 2.8 mg/dL, IL-6 is below 7.8 pg/mL, PDGF-AB BB is below 10,443 pg/mL, and/or VEGF-A is below 77.5 pg/mL.

In one embodiment the subject is determined to have an FRI, if at least one of CRP, IL-6, PDGF-AB BB, and VEGF-A is above the indicated level, with the sensitivity being at least about 84 and specificity being at least about 69, or when at least two of CRP, IL-6, PDGF-AB BB, and VEGF-A are above the indicated levels, with the sensitivity being at least about 61 and specificity being at least about 76, or when at least three of CRP, IL-6, PDGF-AB BB, and VEGF-A are above the indicated levels, with the sensitivity being at least about 38 and specificity being at least about 92, or when all four of CRP, IL-6, PDGF-AB BB, and VEGF-A are above the indicated levels, with the sensitivity being at least about 23 and specificity being about 100. In one embodiment monokine induced by gamma interferon (MIG) are also measured, wherein elevated levels of MIGs relative to a control sample from a healthy patient indicates that the subject likely has an FRI.

In accordance with one embodiment the methods disclosed herein for detecting FRI in subjects can include one or more parameters selected from fracture region, number of fractures, gender, age, and underlying systemic inflammation diseases.

In accordance with embodiment 1, a method of measuring a spectroscopic profile of a subject's biological sample is provided wherein the method comprises obtaining a test spectroscopic profile of a biological sample obtained from a subject wherein absorbance is measured at any one of
i) about 1624-1625 cm⁻¹;
ii) about 1188-1189 cm⁻¹;
iii) about 610-611 cm⁻¹;
iv) about 1592-1593 cm⁻¹;
v) about 1648-1649 cm⁻¹; or
vi) about 3288-3289 cm⁻¹, or any combination thereof, optionally
   wherein the biological sample is a urine or blood component, optionally wherein the blood component is selected from plasma or serum.

In accordance with embodiment 2, a method of detecting a patient's biological sample that exhibits two or more of the following:
ii) i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL is provided wherein said method comprises
obtaining a plasma sample from a human patient; and
detecting CRP, IL6, PDGF-AB BB and VEGF-A present in the plasma sample, by contacting the plasma sample with antibodies specific for the corresponding CRP, IL6, PDGF-AB BB and VEGF-A, and detecting binding between said antibodies and their target protein, optionally wherein the biological sample is a urine or blood component, optionally wherein the blood component is selected from plasma or serum.

In accordance with embodiment 3 a method of diagnosing a patient with an FRI is provided where the method comprises determining if a subject's biological sample exhibits a biomarker associated with FRI, wherein the biomarker is
i) a spectroscopic profile of a plasma sample associated with FRI or
ii) an increased relative expression of one or more proteins selected from the group consisting of interleukin 6 (IL-6), platelet-derived growth factor AB BB (PDGF-AB BB), and vascular endothelial growth factor A (VEGF-A), optionally wherein the biological sample is a urine or blood component, optionally wherein the blood component is selected from plasma or serum.

In accordance with embodiment 4 a method of any one of embodiments 1-3 is provided wherein the biological sample is a plasma sample, and the steps of identifying a spectroscopic profile of a plasma sample associated with FRI comprises:
obtaining a test spectroscopic profile of a plasma sample obtained from the subject; and comparing the test spectroscopic profile to a control spectroscopic profile of a plasma sample,
wherein higher absorbance at about 1624-1625 cm⁻¹ and/or about 1188-1189 cm⁻¹ and/or lower absorbance at about 610-611 cm⁻¹, 1592-1593 cm-1, 1648-1649 cm⁻¹, and/or 3288-3289 cm⁻¹ of the test spectroscopic profile when compared to the control spectroscopic profile indicates that the subject has an FRI.

In accordance with embodiment 5 a method of any one of embodiments 1-4 is provided wherein the steps of identifying elevated levels of one or more proteins selected from the group consisting of interleukin 6 (IL-6), platelet-derived growth factor AB BB (PDGF-AB BB), and vascular endothelial growth factor A (VEGF-A) comprises:
obtaining a test protein biomarker profile of a plasma sample obtained from said test subject; and comparing the test protein biomarker profile to a control protein biomarker profile of a plasma sample,
wherein elevated expression of at least one of the proteins in the test protein biomarker profile relative to the control protein biomarker profile identifies a patient having an FRI.

In accordance with embodiment 6 a method of any one of embodiments 1-5 is provided wherein the steps of determining if the subject has elevated levels of said three or more proteins comprises detecting at least three of the following:
i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL in a plasma sample form said subject.

In accordance with embodiment 7 a method of any one of embodiments 1-6 is provided wherein said proteins are quantified using an antibody that specifically binds to the respective proteins.

In accordance with embodiment 8 a method of treating a fracture-related infection (FRI) in a subject having FRI is provided wherein the method comprises:
analyzing a subject's plasma sample to detect a biomarker associated with FRI, and identifying a subject having an FRI, wherein the biomarker is
   i) a spectroscopic profile of a plasma sample associated with FRI or
   ii) an increased relative expression of one or more proteins selected from a test profile comprising interleukin 6 (IL-6), platelet-derived growth factor AB BB (PDGF-AB BB), and vascular endothelial growth factor A (VEGF-A); and
administering to said subject identified as having an FRI an anti-infection therapy, optionally wherein the anti-infection therapy is the administration of antibiotics.

In accordance with embodiment 9 a method of any one of embodiments 1-8 is provided wherein the steps of identifying a spectroscopic profile of a plasma sample associated with FRI comprises:
obtaining a test spectroscopic profile of a plasma sample obtained from the subject; and comparing the test spectroscopic profile to a control spectroscopic profile of a plasma sample,
wherein higher absorbance at
   i) about 1624-1625 cm⁻¹;
   ii) about 1188-1189 cm⁻¹; or
   iii) a combination of i) and ii) and/or
lower absorbance at
   iv) about 610-611 cm⁻¹;
   v) about 1592-1593 cm⁻¹;
   vi) about 1648-1649 cm⁻¹;
   vii) about 3288-3289 cm⁻¹ or
   viii) or any combination of iv-vii
      of the test spectroscopic profile when compared to the control spectroscopic profile indicates that the subject has an FRI.

In accordance with embodiment 10 a method of any one of embodiments 1-9 is provided wherein the higher absorbance is at
i) about 1624.3 cm⁻¹;
ii) about 1188.2 cm⁻¹; or
iii) a combination of i) and ii) and/or the lower absorbance is at
iv) about 610.6 cm⁻¹;
v) about 1592.9 cm⁻¹;
vi) about 1648.6 cm⁻¹;
vii) about 3288.7 cm⁻¹ or
viii) or any combination of iv-vii.

In accordance with embodiment 11 a method of any one of embodiments 1-10 is provided wherein the higher absorbance is at
about 1624.3 cm⁻¹; and about 1188.2 cm⁻¹; and the lower absorbance is at 610.6 cm-1, 1592.9 cm-1, 1648.6 cm⁻¹, and 3288.7 cm-1.

In accordance with embodiment 12 a method of any one of embodiments 1-11 is provided wherein the spectrometer is an FTIR spectrometer.

In accordance with embodiment 13 a method of any one of embodiments 1-12 is provided wherein the steps of identifying elevated levels of one or more proteins selected from the test profile comprises:
determining the expression profile of one or more proteins selected from said test profile in a plasma sample obtained from said test subject relative to the corresponding expression profile of one or more proteins selected from said test profile in a plasma sample obtained from a control plasma sample,
wherein elevated expression of at least one of the proteins in the test protein biomarker profile in the test subject's plasma sample relative to the control plasma sample identifies a patient having an FRI.

In accordance with embodiment 14 a method of any one of embodiments 1-13 is provided wherein the plasma sample is analyzed for elevated expression of two or more proteins selected from the group consisting of C-reactive protein (CRP), IL-6, PDGF-AB BB and VEGF-A.

In accordance with embodiment 15 a method of any one of embodiments 1-14 is provided wherein the plasma sample is analyzed for:
i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL wherein detection of any two of i), ii), iii) and
iv) identifies a subject having an FRI.

In accordance with embodiment 16 a method of any one of embodiments 1-15 is provided wherein the plasma sample is analyzed for:
i) a C-reactive protein (CRP) plasma concentration above about 2.8 mg/dL;
ii) an interleukin 6 (IL-6) plasma concentration above about 7.8 pg/mL;
iii) a platelet-derived growth factor-AB BB (PDGF-AB BB) plasma concentration above about 10,443 pg/mL; or
iv) a vascular endothelial growth factor A (VEGF-A) plasma concentration above about 77.5 pg/mL wherein detection of any two of i), ii), iii) and iv) identifies as a subject having an FRI.

In accordance with embodiment 17 a method of any one of embodiments 1-16 is provided wherein said proteins are quantified using an antibody that specifically binds to the respective proteins.

In accordance with embodiment 18 a method of any one of embodiments 15-17 is provided wherein three of i) through iv) are detected.

In accordance with embodiment 19 a method of any one of embodiments 1-11 is provided wherein the detection of CRP above 2.8 mg/dL, IL-6 above 7.8 pg/mL, PDGF-AB BB above 10,443 pg/mL, and VEGF-A above 77.5 pg/mL identifies as a subject having an FRI.

In accordance with embodiment 20 a method of any one of embodiments 1-19 is provided, further comprising the step of measuring monokine induced by gamma interferon (MIG) in a subjects plasma, wherein detected elevated levels of MIG relative to a control sample indicates a subject having an FRI.

In accordance with embodiment 21 a method of treating a fracture-related infection (FRI) in a subject having FRI, said method comprising:
identifying a subject having elevated levels of three or more proteins selected from the group consisting of C-reactive protein (CRP), interleukin 6 (IL-6), platelet-derived growth factor AB BB (PDGF-AB BB), and vascular endothelial growth factor A (VEGF-A), as a subject having an FRI; and
administering to said subject identified having an FRI, an anti-infection therapy.

### EXAMPLES

The following examples serve to illustrate the present disclosure. The examples are not intended to limit the scope of the claimed invention.

### Example 1. Four Biomarkers Accurately Differentiated FRI Patients from Matched Control Patients

### 1A. Patient Selection Methodology

The study was performed over a period of nine months. Inclusion criteria for both the confirmed FRI and control groups were age 18 to 85 years and an extremity, pelvic ring, or acetabulum fracture that was surgically treated with retained orthopaedic implant within the last two years. Additionally, specifically for the confirmed FRI group, a clinically suspected FRI was required for inclusion. Exclusion criteria included the following: hand and spine fractures, pregnancy, incarceration, known immunosuppressive state (e.g., lupus, cancer, human immunodeficiency virus (HIV), hepatitis C, rheumatologic disease, or any patient taking an immune-modulating medication), known separate source infection (e.g., urinary tract infection, pneumonia, decubitus ulcer), systemic infection (e.g., sepsis or bacteremia), undergoing second debridement or prior failed infection treatment, pathologic fracture, definitive treatment without retained implant (i.e., arthroplasty, percutaneous Kirschner wires, or external fixation), known venous thromboembolism, and hemodialysis. All FRI confirmed patients were enrolled in the study and blood samples were obtained prior to surgical intervention for treatment of the infected site. Non-infected control patients were identified and matched to the FRI patients based on age (±15 years), time after surgery (±2 weeks), and fracture region. Fracture regions were matched as follows: upper extremity long bones (humerus, radius, and ulna), lower extremity long bones (femur and tibia), and other lower extremity bones (e.g., patella, tarsal bones of the foot). Control patients were identified through routine clinical follow-up. All controls remained infection-free for a minimum of 6 months after enrollment as determined by routine clinic follow-up, chart review, or phone calls.

### 1B. Patient Cohort Data

Eighty-two patients were screened for enrollment. Thirty-two patients were screen failures (alternative source of infection (n=3), pathologic fracture (n=3), thromboembolic disease (n=1), age<18 (n=2), immunosuppressive state (n=5), unable to provide consent (n=1), HIV+ (n=1), undergoing second treatment or prior failure of infection treatment (n=13), definitive management including percutaneous Kirshner wires or external fixation pins (n=1), and hemodialysis (n=2)), 8 declined to participate, and 2 were unable to be enrolled due to difficulty collecting blood samples. Ultimately, 40 patients were enrolled in the study and had blood samples collected. Two cases were later withdrawn after enrollment; one due to discovery of prior nonunion surgery and the other due to detection of a separate infectious source (i.e., urinary tract infection). The final cohort included 22 confirmed FRIs and 16 controls. Using the above-described matching criteria, 13 pairs of confirmed FRIs were matched with controls. All 13 of the FRIs met confirmatory criteria with either fistula/sinus/wound breakdown and/or purulent drainage on initial presentation. Eight of 13 (62%) had at least 2 positive cultures with phenotypically indistinguishable pathogens from their infection surgery.

Table 1 summarizes patient demographic, clinical, and co-morbidity data for FRIs and controls. The mean enrollment time was 6 weeks post-operative, with 69% (18/26) of the entire cohort having femur or tibia bone involvement, 54% (14/26) having plate fixation, and 15% (4/26) having an open fracture. As Table 1 shows, there was no significant differences in age or fracture region.

TABLE 1
Description of Cohort*

| | | | |
|---|---|---|---|
| Overall Cohort | FRI | Control | |
| (n = 26) | (n = 13) | (n = 13) | p-value** |

Demographics

| Age | 51.3 | (14.9) | 51.4 | (14.8) | 51.2 | (15.5) | 0.953 |
|---|---|---|---|---|---|---|---|
| Sex | | | | | | | 0.226 |
| Male | 16 | | 10 | | 6 | | |
| Female | 10 | | 3 | | 7 | | |
| BMI | 30.2 | (7.9) | 28 | (4.6) | 32.4 | (9.9) | 0.209 |
| Weeks Post-Operation | 6.0 | (4.3) | 6.4 | (4.5) | 5.5 | (4.4) | 0.043 |

Clinical

| Bone Involvement | | | | >0.999 |
|---|---|---|---|---|
| Femur/Tibia | 18 | 9 | 9 | |
| Patella/Ankle/Foot | 6 | 3 | 3 | |
| Upper Extremity/Clavicle | 2 | 1 | 1 | |
| Implant Used | | | | 0.047 |
| IMN | 12 | 9 | 3 | |
| Plate | 14 | 4 | 10 | |
| Fracture Type | | | | 0.096 |
| Open | 4 | 4 | 0 | |
| Closed | 22 | 9 | 13 | |
| NSAID/Steroid Use | | | | >0.999 |
| Yes | 1 | 1 | 0 | |
| No | 25 | 12 | 13 | |

Co-morbidities

| Diabetes Mellitus | | | | 0.322 |
|---|---|---|---|---|
| Yes | 5 | 1 | 4 | |
| No | 21 | 12 | 9 | |
| History of MRSA | | | | 0.48 |
| Yes | 2 | 2 | 0 | |
| No | 24 | 11 | 13 | |
| Tobacco Use | | | | >0.999 |
| Yes | 7 | 4 | 3 | |
| No | 19 | 9 | 10 | |
| Alcohol Abuse | | | | 0.48 |
| Yes | 2 | 2 | 0 | |
| No | 24 | 11 | 13 | |

*Values are means (S.D.) for continuous data (i.e., age, BMI, weeks post-operation). All other values are counts.
**Result from two-sided matched t-test for continuous data and Fisher's Exact test for categorical data.

1C. Collecting and Processing Blood from Patients

ESR, CRP, and WBC, as well as three intra-operative cultures and gram stains, were obtained as part of the standard of care for the FRI patients. Peripheral blood samples were obtained from the FRI cohort on the day of surgery for infection. Specifically, an EDTA purple top tube (BD Vacutainer®, Becton, Dickinson and Company, Franklin Lakes, NJ) was utilized for collection of approximately 5 mL of whole blood. The tube was inverted 4-5 times to allow the blood to mix with the anticoagulant. The tube was placed and balanced in a table-top centrifuge and spun at 1500 g for 10 minutes with acceleration and deceleration set at 9. This default setting allows the centrifuge to reach not only the set centrifugal force (1500 g) but also brake or decelerate in the shortest time following the spin. Plasma was then extracted, aliquoted into 500 microliter tubes, and stored at −80° C. Blood samples for controls were collected from patients during routine post-operative clinic visits with the same collection and processing as described above.

### 1D. Plasma Protein Selection and Measurements

An assay kit was used for protein multiplex ELISA (EMD Millipore Corporation, Burlington, MA). The panel contained the following 48 proteins: soluble CD40L (sCD40L), Epidermal growth factor (EGF), Eotaxin, basic fibroblast growth factor 2 (FGF-2), Fms-related tyrosine kinase 3 ligand (FLT-3L), Fractalkine, Granulocyte colony-stimulating factor 3 (G-CSF), Granulocyte macrophage colony-stimulating factor GM-CSF, C—X—C motif ligand 1 (CXCL1), C—X—C motif chemokine ligand 10 (CXCL10), Interferons selected from: IFNα2, IFNγ, interleukins selected from: IL1α, IL-1β, IL-1RA, IL-2, IL-3, IL-4, IL-5, IL-6, IL-7, IL-8, IL-9, IL-10, IL-12 (p40), IL-12 (p70), IL-13, IL-15, IL-17A, IL-17E/IL-25, IL-17F, IL-18, IL-22, IL-27, Monocyte Chemoattractant Protein-1 (MCP-1), Monocyte Chemoattractant Protein-3 (MCP-3), Macrophage colony-stimulating factor (M-CSF), Macrophage-derived chemokine (MDC), Monokine induced by gamma (MIG), Macrophage inflammatory protein-1 alpha (MIP-1α), Macrophage inflammatory protein-1 beta (MIP-1β), Platelet Derived Growth Factor-AA (PDGF-AA), Platelet Derived Growth Factor-AB/BB (PDGF-AB BB), RANTES, transforming growth factor α (TGFα), TNFα, transforming growth factor β (TGFβ), and endothelial growth factor A (VEGF-A). This immunoassay was selected because it contains a substantial number of relevant inflammatory biomarkers that have been associated with the inflammatory response to infection. Additionally, CRP levels were measured on all samples. For samples with biomarker concentrations that were undetectable, (½*lowest detectable value) was used for data analysis. Any samples that exceeded the maximal detectable value were diluted, re-measured, and corrected for dilution.

### 1E. Plasma Sample Analysis

Of the 49 plasma proteins assessed, seven trended (p<0.1) towards being significantly different between the FRI and control groups, with 4 of these having p<0.05. Tables 2 and 3 show the plasma concentrations for all proteins assessed. As IL-6, PDGF-AB BB, VEGF-A, and CRP were significantly different between the two groups, they were carried forward into ROC curve analyses. Cut-points optimizing the ROC analyses were 7.8 pg/mL, 10,443 pg/mL, 77.5 pg/mL for IL-6, PDGF-AB BB, and VEGF-A, respectively. Cut point was 2.8 mg/dL for CRP. Areas under the curve (AUCs) for these cut points ranged from 0.654-0.731 (See Table 4). Examined cumulatively, having all four of these biomarkers below their respective cut-points was 100% specific for FRI (See Table 5).

TABLE 2
Statistically Significant Biomarkers

| Biomarker* | FRI (n = 13) | | Control (n = 13) | | p-value** |
|---|---|---|---|---|---|
| CRP | 5.61 | (6.92) | 1.17 | (1.36) | 0.046 |
| IL-6 | 26.45 | (35.36) | 3.67 | (2.57) | 0.036 |
| PDGF-AB BB | 15561 | (8926) | 7773 | (2656) | 0.023 |
| VEGF-A | 98.88 | (96.56) | 37.36 | (18.33) | 0.033 |

*Values are means (S.D.) for concentration of biomarker in plasma. Units are mg/dL for CRP and pg/mL for all others.
**Result from two-sided matched t-test.

TABLE 3
Other Biomarkers*

| | | | Detectable | | | Detectable | p- |
|---|---|---|---|---|---|---|---|
| Biomarker | FRI | | (n)** | Control | | (n) | value*** |
| MCP-1 | 275.97 | (125.44) | 13 | 275.70 | (106.88) | 13 | 0.996 |
| MIP-1a | 18.52 | (29.93) | 6 | 19.33 | (16.63) | 8 | 0.943 |
| IL-3 | 0.53 | (0.85) | 2 | 0.57 | (1.21) | 2 | 0.932 |
| IL-12(P40) | 70.39 | (52.41) | 13 | 68.37 | (41.01) | 13 | 0.914 |
| IL-17F | 39.89 | (119.44) | 3 | 34.40 | (80.04) | 8 | 0.899 |
| TGFa | 5.99 | (17.58) | 4 | 5.18 | (11.71) | 8 | 0.899 |
| M-CSF | 88.16 | (123.20) | 10 | 97.89 | (112.53) | 8 | 0.856 |
| IL-13 | 26.49 | (37.94) | 9 | 23.54 | (14.79) | 10 | 0.772 |
| IL-10 | 9.28 | (12.16) | 10 | 8.06 | (8.3) | 9 | 0.757 |
| IL-22 | 31.03 | (82.84) | 3 | 23.07 | (27.10) | 5 | 0.718 |
| IL-1a | 13.63 | (33.58) | 9 | 9.27 | (7.23) | 12 | 0.679 |
| FLT-3L | 14.05 | (10.68) | 13 | 15.93 | (6.50) | 13 | 0.651 |
| IL-1RA | 8.29 | (8.19) | 13 | 10.18 | (8.99) | 13 | 0.607 |
| IFNa2 | 44.99 | (52.47) | 11 | 33.98 | (28.92) | 12 | 0.571 |
| MCP-3 | 22.97 | (38.66) | 10 | 16.55 | (7.79) | 13 | 0.553 |
| IL-1b | 12.74 | (36.93) | 7 | 6.05 | (6.61) | 10 | 0.551 |
| IL-8 | 4.24 | (2.37) | 13 | 3.80 | (1.94) | 13 | 0.528 |
| TNFa | 28.06 | (17.30) | 13 | 24.61 | (7.66) | 13 | 0.526 |
| IL-2 | 1.11 93.04) | | 4 | 0.50 | (0.71) | 7 | 0.518 |
| IL-17E IL-25 | 251.70 | (688.11) | 5 | 388.53 | (265.77) | 12 | 0.504 |
| IL-4 | 2.40 | (4.79) | 8 | 1.44 | (1.96) | 10 | 0.500 |
| IL-17A | 7.87 | (21.83) | 7 | 2.78 | (2.62) | 9 | 0.436 |
| FGF-2 | 98.09 | (195.80) | 13 | 53.53 | (17.96) | 13 | 0.434 |
| Fractalkine | 151.08 | (188.64) | 12 | 103.26 | (60.92) | 12 | 0.390 |
| GM-CSF | 3.19 | (2.95) | 1 | 2.42 | (0.84) | 1 | 0.387 |
| IP-10 | 231.20 | (204.91) | 13 | 173.65 | (69.33) | 13 | 0.376 |
| IL-18 | 30.00 | (33.84) | 12 | 40.44 | (21.69) | 13 | 0.376 |
| G-CSF | 66.81 | (73.73) | 11 | 47.63 | (38.92) | 11 | 0.368 |
| IL-5 | 5.08 | (4.19) | 13 | 7.22 | (5.50) | 13 | 0.345 |
| TNFb | 3.66 | (9.53) | 7 | 1.08 | (1.74) | 7 | 0.327 |
| MIP-1b | 32.42 | (48.67) | 13 | 17.94 | (5.79) | 13 | 0.314 |
| IL-12(P70) | 9.89 | (30.34) | 7 | 1.28 | (2.00) | 5 | 0.312 |
| Eotaxin | 53.25 | (34.96) | 13 | 70.38 | (39.74) | 13 | 0.281 |
| IFNr | 7.70 | (19.63) | 9 | 1.43 | (1.18) | 12 | 0.268 |
| Rantes | 94985 | (58356) | 13 | 74073 | (28388) | 13 | 0.264 |
| IL-15 | 7.50 | (4.59) | 13 | 12.57 | (12.85) | 13 | 0.263 |
| IL-9 | 9.76 | (12.82) | 10 | 17.50 | (15.83) | 12 | 0.262 |
| IL-7 | 1.76 | (2.10) | 9 | 1.07 | (1.35) | 8 | 0.239 |
| GROa | 16.88 | (16.19) | 9 | 10.50 | (8.03) | 9 | 0.230 |
| MIG CXCL9 | 2673.6 | (2636.5) | 13 | 1535.8 | (665.41) | 13 | 0.184 |
| IL-27 | 2775.4 | (1938.1) | 13 | 1840.5 | (983.3) | 13 | 0.149 |
| EGF | 47.68 | (61.93) | 11 | 16.90 | (10.01) | 12 | 0.117 |
| sCD40L | 512.78 | (472.19) | 13 | 242.57 | (121.53) | 13 | 0.078 |
| MDC | 754.8 | (196.09) | 13 | 1057.4 | (534.13) | 13 | 0.075 |
| PDGF-AA | 1278.1 | (962.86) | 13 | 633.15 | (342.35) | 13 | 0.068 |

*Values are means (S.D.) for concentration of biomarker in plasma. Units are in pg/mL.
**Not all patients had detectable levels of each biomarker. The number in this column shows how many patients had a detectable level.
***Result from two-sided matched t-test.

TABLE 4
ROC Optimization

| Biomarker | Cut-Point | | Sensitivity | Specificity | AUC* |
|---|---|---|---|---|---|
| CRP | 2.8 | mg/dL | 46.2 | 92.3 | 0.692 |
| IL-6 | 7.8 | pg/mL | 53.9 | 84.6 | 0.692 |
| PDGF-AB BB | 10,443 | pg/mL | 61.5 | 84.6 | 0.731 |
| VEGF-A | 77.5 | pg/mL | 38.5 | 92.3 | 0.654 |

*AUC = Area under the curve. Logistic regression models were performed to determine the AUC, as well as to derive the cut-points for the Youden index.

TABLE 5
Cumulative Diagnostic Parameters for
CRP, IL-6, PDGF-AB BB, and VEGF-A

| Markers above cut-point | Sensitivity | Specificity | AUC* |
|---|---|---|---|
| At least 1 of the 4 | 84.6 | 69.2 | .769 |
| At least 2 of the 4 | 61.5 | 76.9 | .692 |
| At least 3 of the 4 | 38.5 | 92.3 | .654 |
| At least 4 of the 4 | 23.1 | 100 | .615 |

*AUC = Area under the curve. Logistic regression models were performed to determine the AUC, as well as to derive the cut-points for the Youden index.

### 1F. Statistical Analysis

Plasma protein differences between the FRI and control groups were assessed using two-sided matched t-tests. Although change/paired data are typically linear, plasma protein results can be skewed, so non-parametric signed rank tests were also performed to verify the results of the paired t-tests (similar findings, results not shown).

To analyze the predictability of plasma proteins to categorize treatment group participants, logistic regression models were performed, and ROC curves were generated to determine the optimal cut-points for each protein, using the Youden J Index for this optimal cut-point. A cumulative index, ranging from 0 to 4, for the four significant (p<0.05) proteins was also calculated by summing the number of proteins that were above separate Youden values, and ROC analyses were performed on each category (having at least one, having at least two, etc.) in order to determine if this can give a more accurate prediction. All analytic assumptions were verified, and all analyses were performed using SAS v9.4 (SAS Institute, Cary, NC).

For multivariate analysis, both data sets (protein measurements and MIR spectra) were imported into the MATLAB® software (MathWorks R2015b (8.6.0.267246), Natick, MA). In-house written scripts were utilized for processing. Initially, spectral data were smoothed using the Savitsky-Golay filter (2^{nd} degree polynomial functions and 11-point smoothing window). Standard normal variate transform (SNV) and baseline normalization to the KSCN peak were used for spectral normalization. Verification of whether an observation was an outlier or not relied on the values of the two statistics, T2 and Q, for both of which the null hypothesis was tested at a 95% confidence level. The average of the three replicates for each sample was used for analysis. Statistical significance was set at P<0.05.

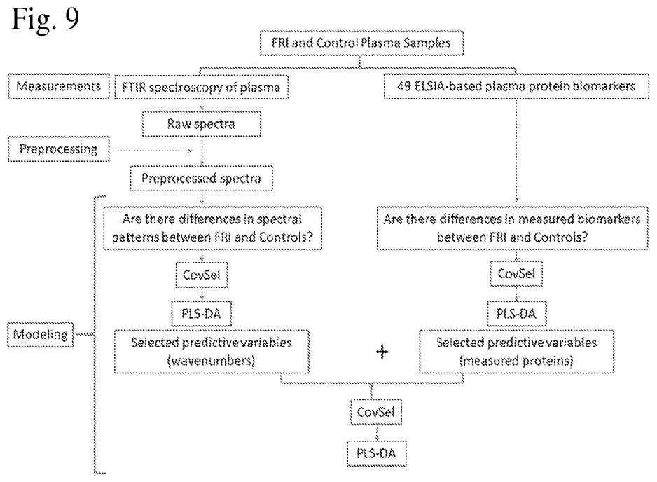

To allow comparison of the utility of ELISA-based proteins and sample spectral patterns as predictors of FRI, both data sets were used to build multivariate classification models to discriminate between FRI and control samples, with subsequent validation. Partial least squares discriminant analysis (PLS-DA) was used for classification to address the low number of patient samples in the training set compared to the number of measured variables for both data sets. In order to improve classification accuracy and to identify a minimum set of proteins and spectral wavenumbers out of the whole set of variables, the PLS-DA classification algorithm was coupled with covariance selection (CovSel). The CovSel-PLS-DA model was built and validated through a repeated double-cross-validation (rDCV) procedure with 13 segments in the outer loop and 12 in the inner loop using 50 repetitions. For each cancelation group in the outer loop, predictions were carried out on a model built on the remaining samples. The best subset of original variables to be used as inputs and the optimal number of latent variables were selected as those leading to the minimum classification error in the inner-loop cross-validation procedure. Data were auto-scaled prior to the analysis. Lastly, the selected variables from the two platforms were integrated in a mid-level data fusion approach. The predictors were auto-scaled, and the proteins and MIR spectra data were further block-scaled to allow equal contributions. For each comparison the accuracy, sensitivity, and specificity of the predictive model, as well as the AUC of the ROC curve, are reported as measures of the model's performance. Exemplary steps of multivariate analysis are summarized in FIG. 9.

### Example 2. Quick Spectroscopy Analysis of Plasma has a Similar Sensitivity and Accuracy as an ELISA

ELISA is the gold standard for identifying the presence and relative expression levels of biomolecules. However, ELISA is expensive and is not useful as a quick diagnostic method. The following exemplifies that the disclosed method is similarly sensitive and accurate as the antibody test with the added advantage of being substantially cheaper and providing results faster.

### 2A. Infrared Spectroscopy

Thawed plasma samples were diluted with potassium thiocyanate (KSCN, SigmaUltra, Sigma-Aldrich Inc, St Louis, MO) as an internal control in a 2:1 ratio. Using a previously described technique, three 8 L replicates of each sample were applied on a 96-welled silicon microplate and allowed to dry at room temperature (20-22° C.). Each microplate was placed in the multi-sampler (HTS-XT, Bruker Scientific, LLC, Billerica, MA, USA) attachment of an FTIR spectrometer (INVENIO S, Bruker Scientific, LLC, Billerica, MA, USA). Mid-IR (MIR) absorbance spectra in the wavenumber range of 400 to 4,000 cm⁻¹ was recorded using the OPUS software (version 6.5, Bruker Optics, GmbH, Ettlingen, Germany). For each sample evaluation, 512 interferograms were signal averaged and Fourier-transformed to produce a nominal resolution of 4 cm⁻¹ for the resulting spectrum.

### 2B. Multivariate Analysis of Biomarkers

A multivariate analysis-based predictive model developed utilizing ELISA-based biomarkers had sensitivity, specificity, and accuracy of 69.2±0.0%, 99.9±1.0%, and 84.5±0.6%, respectively, with PDGF-AB/BB, CRP, and MIG (i.e., CXCL9) selected as the minimum number of variables explaining group differences. Sensitivity, specificity, and accuracy of the predictive model based on MIR spectra were 69.9±6.2%, 71.9±5.9%, and 70.9±4.8%, respectively, with six wavenumbers as explanatory variables (3288.7, 1648.6, 1624.3, 1592.9, 1188.2 and 610.6 cm⁻¹).

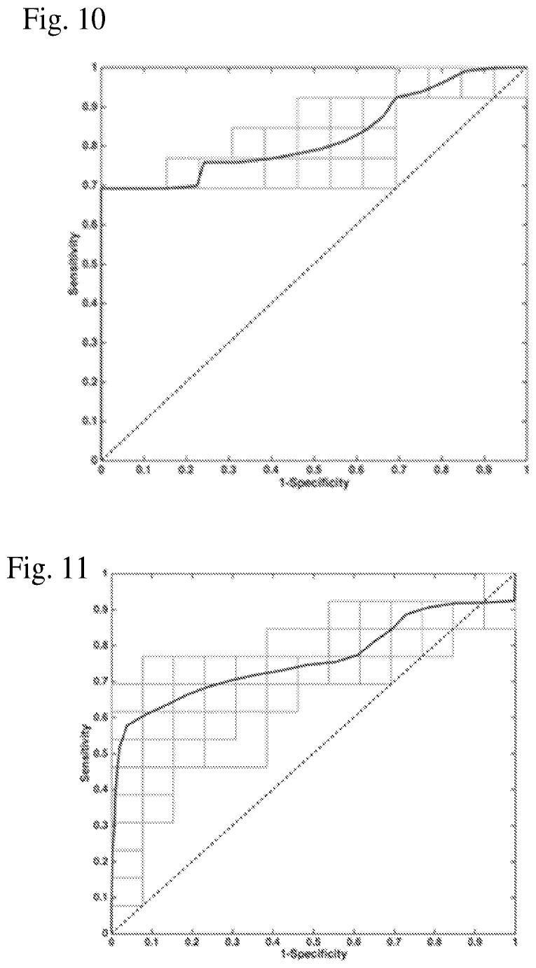

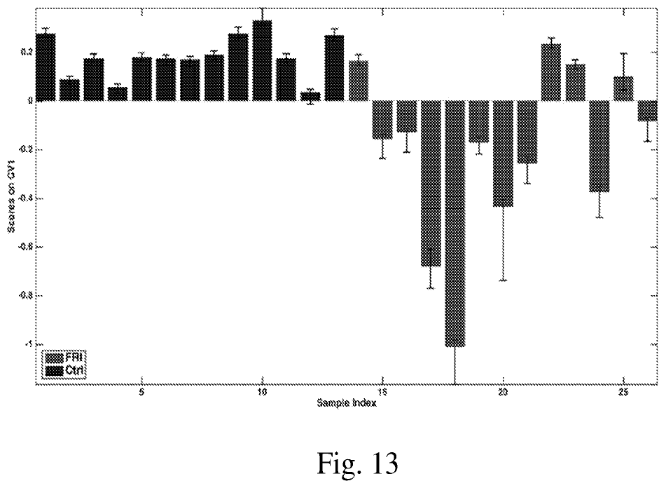

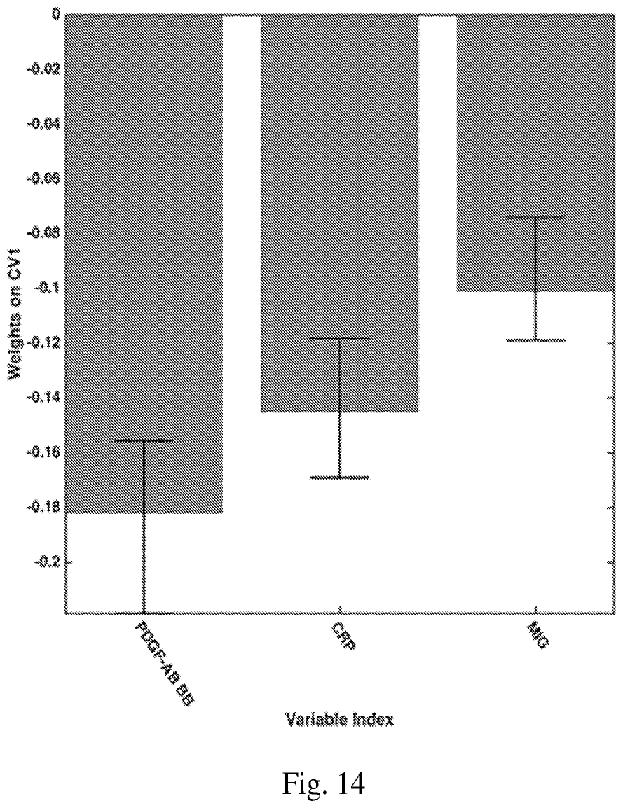

For multivariate analysis using plasma protein levels as predictors resulted in PDGF-AB/BB, CRP, and MIG being retained to build the classification model. The overall classification accuracy on the external loop samples was found to be 84.5±0.6%, with 69.2±0.0% sensitivity and 99.9±1.0% specificity (FIG. 10). The AUC for this model was 0.826±0.018, demonstrating an excellent discriminant ability. The visual interpretation of the results based on comparing the scores of the outer loop samples along the only canonical variate of the model with the variable weights for the construction of that direction is presented in FIG. 13 and FIG. 14.

### 2C. FTIR Spectroscopy

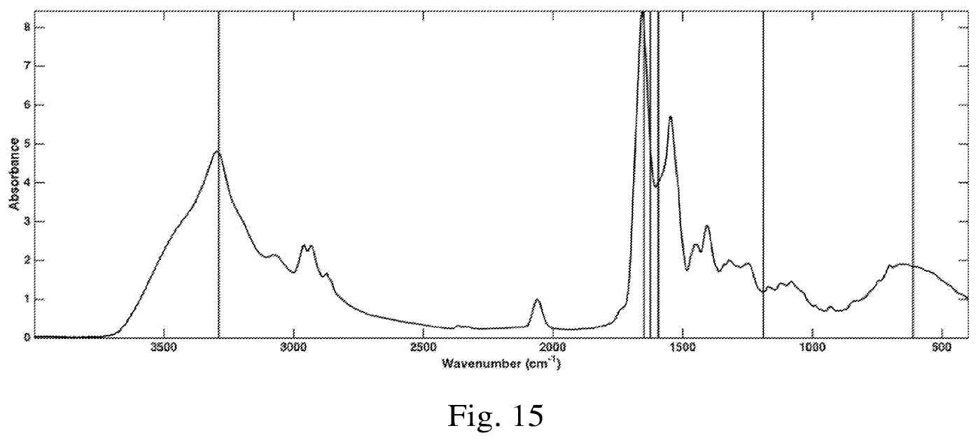

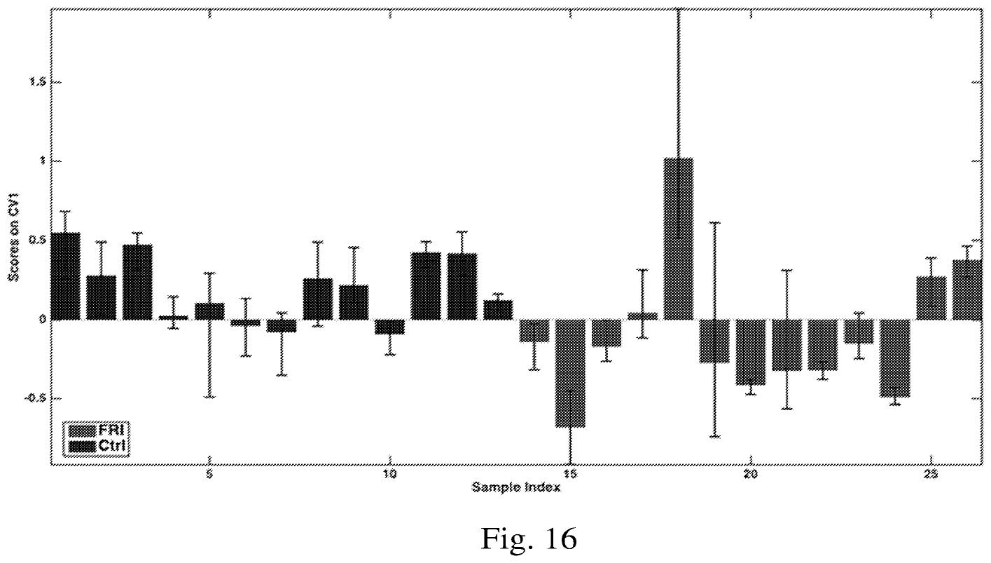

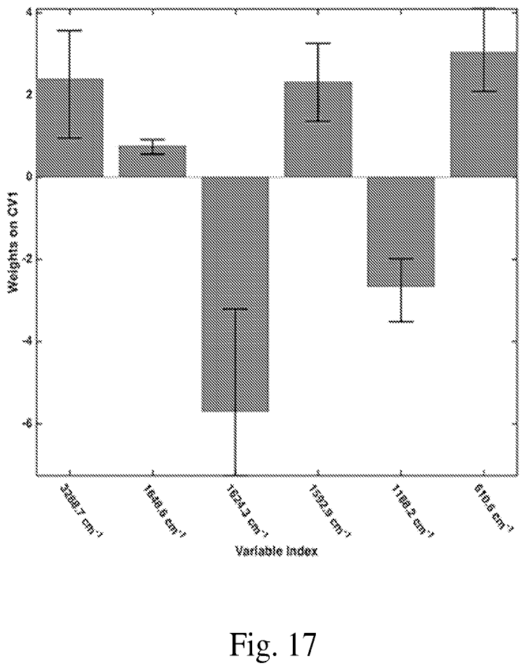

Analysis of the FTIR spectroscopic data, during the model-building phase, resulted in six latent variables (i.e., the six wavenumbers from Example 2B) as optimal complexity (FIG. 15). The overall classification accuracy on the outer loop samples (as defined by the CovSel-PLS-DA model) was found to be 70.9±4.8%, with 69.9±6.2% sensitivity and 71.9±5.9% specificity (FIG. 11). The AUC for this model was 0.761±0.041 indicating an acceptable discriminant ability. Permutation test confirmed that the observed figures of merit were all statistically significant (P<0.05). The results based on comparing the scores of the outer loop samples along the only canonical variate of the model with the variable weights for the construction of that direction are presented in FIG. 16. The scores plot indicated that FRI patients have predominantly negative values, whereas the majority of controls have positive scores. Comparison with the weight values suggests that FRI patients are characterized by higher absorbance at 1624.3 and 1188.2 cm⁻¹ and lower absorbance at 610.6, 1592.9, 1648.6 and 3288.7 cm⁻¹ (FIG. 17).

### 2D. Autoscaling

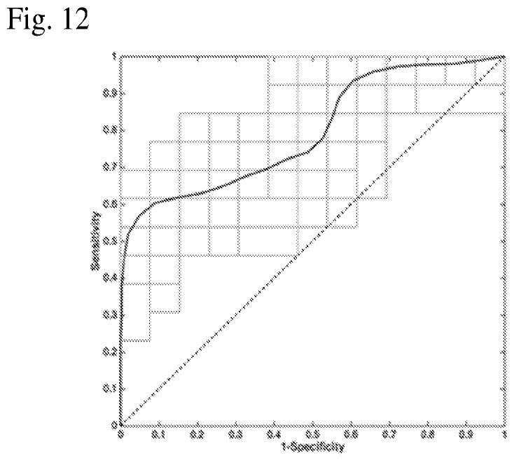

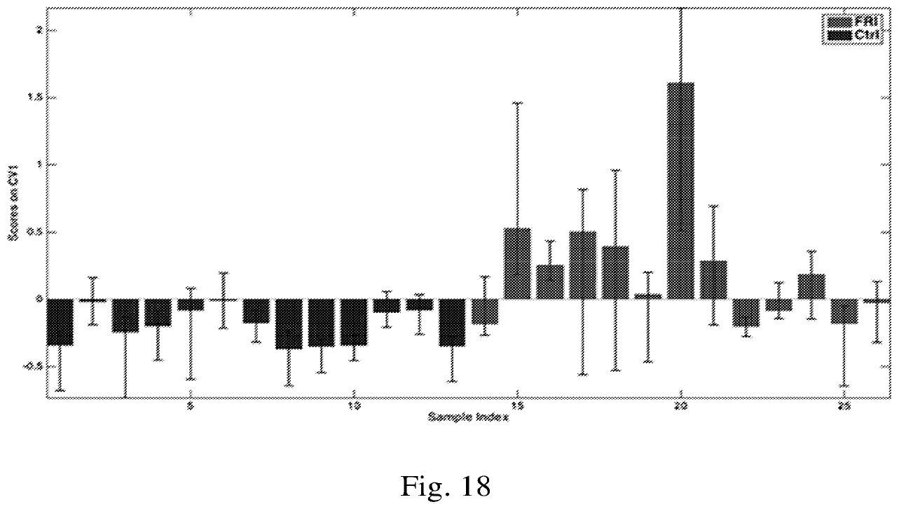

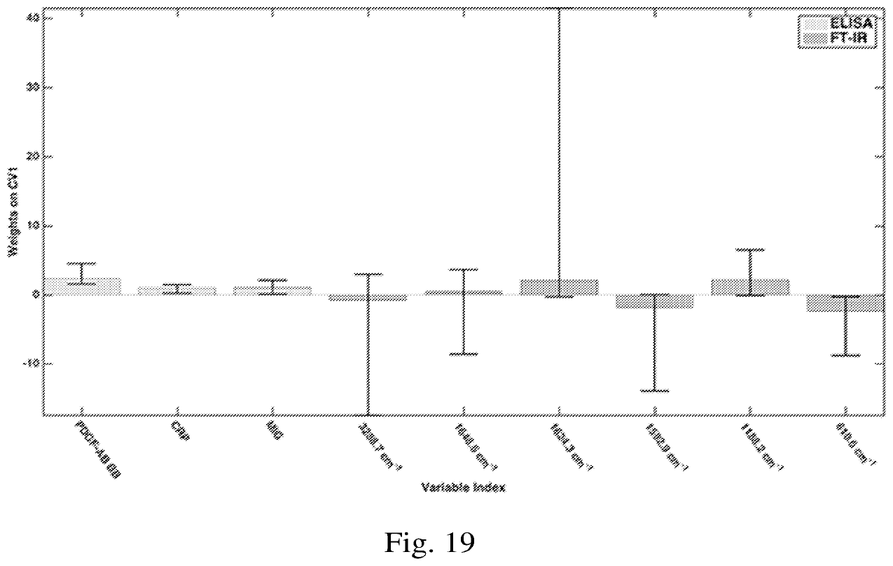

The predictive variables from the previous two Examples (2B and 2C) were autoscaled, and the two blocks of data (proteins and MIR spectra) were further block-scaled to allow equal contributions. The model consistently included four variables (i.e., PDGF-AB/BB, CRP, MIG, and 610.6 cm⁻¹) that contributed significantly to the model (FIG. 18 and FIG. 19) and provided an overall classification accuracy on the external loop samples of 75.2±4.5%, with 61.5±6.3% sensitivity and 88.9±6.6% specificity (FIG. 12). The AUC was 0.795±0.054; indicating near excellent discriminant ability.

### 2E. Discussion

This is the first study demonstrating significant differences comparing measured values of PDGF-AB/BB and VEGF-A between FRI and control patients. It also confirms that CRP and IL-6 may be useful in the diagnostic work-up of FRI.

Complementary approaches of univariate and multivariate analytical methods were used to show biomarker differences from a large panel of inflammatory proteins and MIR spectral signal in plasma obtained from FRI patients and matched controls. Both analytical approaches identified PDGF-AB/BB and CRP as consistent biomarkers with discriminatory abilities. The multivariate method showed MIG combined with PDGF-AB/BB and CRP to be the minimum number of non-redundant variables that significantly contributed to the final predictive model. Univariate analysis identified IL-6 and VEGF-A to be additional biomarkers that were significantly different between groups. The combination of these two analytical methods provides complementary results that reduce loss of information encountered from either analytical approach. Therefore, the results of each analytical approach also require individual interpretation, rather than an attempt to validate the results of one method against the other. The lack of significance for MIG in the univariate approach may be due to an existing covariance of this plasma protein with PDGF-AB/BB and CRP that is identifiable through the multivariate approach. On the other hand, lack of IL-6 and VEGF-A being selected in the multivariate analysis may be due to less significant correlation/covariance between these two and other selected biomarkers. However, this does not imply that these two biomarkers do not have significant differences between the two groups, but that the combination of PDGF-AB/BB, CRP, MIG variables was the minimum number of non-redundant variables that was able to best demonstrate the group differences in the multivariate approach in this cohort.

There is limited literature on novel methods for diagnosis of FRIs. Historically, the standard inflammatory markers used to aide in the diagnosis of FRI have been peripheral WBC, CRP, and ESR. A systematic review of diagnostic accuracy of these “classic” plasma inflammatory markers determined that they are insufficient. In that review, sensitivity and specificity based on CRP ranged from 60-100% and 34-86%, respectively. The ability to predict FRI based on PDGF-AB/BB, CRP, MIG in this study was comparable in sensitivity to previous reports based on CRP alone but significantly improved for specificity and accuracy. The predictive model based on IR spectra variables showed similar sensitivity to that of the select protein biomarkers. However, despite having an acceptable discriminatory ability, the specificity and accuracy were lower than those based on protein biomarkers.

Combining the selected protein biomarkers and spectral variables from each model improved the discriminatory ability of the final predictive model compared to spectral data alone, but it did not surpass the performance of the model based on protein biomarkers alone. These results suggest that, in this cohort of patients, the predictive model based on this select panel of protein biomarkers is the more accurate and specific discriminatory tool, with similar sensitivity compared to spectral fingerprint alone. The comparable results based on MIR spectral data demonstrate the potential ability of this FTIR spectroscopy method to be used as a surrogate for this protein panel as a potential point of care diagnostic screening tool. Advantages of using FTIR spectroscopy of dried films compared to ELISA-based biomarkers include lack of need for adjuvants and cost effectiveness (˜5% the cost of ELISA methods).

The invention claimed is:

1. A method of treating a fracture-related infection (FRI) in a subject having FRI, said method comprising:
analyzing a subject's biological sample to detect a biomarker associated with FRI, and identifying a subject having an FRI, wherein the biomarker is
i) a spectroscopic profile of a biological sample associated with FRI or
ii) an increased relative expression of one or more proteins selected from a test profile comprising interleukin 6 (IL-6), platelet-derived growth factor AB BB (PDGF-AB BB), and vascular endothelial growth factor A (VEGF-A); and
administering to said subject identified as having an FRI an anti-infection therapy, optionally wherein the anti-infection therapy is the administration of antibiotics.

2. The method of claim 1 wherein the biological sample is blood or a blood component, optionally wherein the biological sample is plasma.

4. The method of claim 1 wherein the steps of identifying a spectroscopic profile of a plasma sample associated with FRI comprises:
obtaining a test spectroscopic profile of a plasma sample obtained from the subject; and
comparing the test spectroscopic profile to a control spectroscopic profile of a plasma sample,
wherein higher absorbance at
i) about 1624-1625 cm⁻¹;
ii) about 1188-1189 cm⁻¹; or
iii) a combination of i) and ii) and/or lower absorbance at
iv) about 610-611 cm⁻¹;
v) about 1592-1593 cm⁻¹;
vi) about 1648-1649 cm⁻¹;
vii) about 3288-3289 cm⁻¹ or
viii) or any combination of iv-vii
of the test spectroscopic profile when compared to the control spectroscopic profile indicates that the subject has an FRI.

5. The method of claim 4, wherein the higher absorbance is at
i) about 1624.3 cm⁻¹;
ii) about 1188.2 cm⁻¹; or
iii) a combination of i) and ii) and/or
the lower absorbance is at
iv) about 610.6 cm⁻¹;
v) about 1592.9 cm⁻¹;
vi) about 1648.6 cm⁻¹;
vii) about 3288.7 cm⁻¹ or
viii) or any combination of iv-vii.

6. The method of claim 5, wherein the higher absorbance is at
about 1624.3 cm⁻¹; and about 1188.2 cm⁻¹; and the lower absorbance is at 610.6 cm⁻¹, 1592.9 cm⁻¹, 1648.6 cm⁻¹, and 3288.7 cm⁻¹.

8. The method of claim 2 wherein the steps of identifying elevated levels of one or more proteins selected from the test profile comprises:
determining the expression profile of one or more proteins selected from said test profile in a plasma sample obtained from said test subject relative to the corresponding expression profile of one or more proteins selected from said test profile in a plasma sample obtained from a control plasma sample,
wherein elevated expression of at least one of the proteins in the test protein biomarker profile in the test subject's plasma sample relative to the control plasma sample identifies a patient having an FRI.

9. The method of claim 8 wherein the plasma sample is analyzed for elevated expression of two or more proteins selected from the group consisting of C-reactive protein (CRP), IL-6, PDGF-AB BB and VEGF-A.

10. The method of claim 2 wherein the plasma sample is analyzed for:
i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL wherein detection of any two of i), ii), iii) and iv) identifies a subject having an FRI.

11. The method of claim 2 wherein the plasma sample is analyzed for:
i) a C-reactive protein (CRP) plasma concentration above about 2.8 mg/dL;
ii) an interleukin 6 (IL-6) plasma concentration above about 7.8 pg/mL;
iii) a platelet-derived growth factor-AB BB (PDGF-AB BB) plasma concentration above about 10,443 pg/mL; or
iv) a vascular endothelial growth factor A (VEGF-A) plasma concentration above about 77.5 pg/mL wherein detection of any two of i), ii), iii) and iv) identifies as a subject having an FRI.

12. The method of claim 10 of 11, wherein said proteins are quantified using an antibody that specifically binds to the respective proteins.

13. The method of claim 10, wherein three of i) through iv) are detected.

14. The method of claim 11, wherein detection of CRP above 2.8 mg/dL, IL-6 above 7.8 pg/mL, PDGF-AB BB above 10,443 pg/mL, and VEGF-A above 77.5 pg/mL identifies as a subject having an FRI.

15. The method of claim 1, further comprising the step of measuring monokine induced by gamma interferon (MIG) in a subjects plasma, wherein detected elevated levels of MIG relative to a control sample indicate a subject having an FRI.

16. A method of treating a fracture-related infection (FRI) in a subject having FRI, said method comprising:
I) identifying a subject having elevated levels of three or more proteins selected from the group consisting of C-reactive protein (CRP), interleukin 6 (IL-6), platelet-derived growth factor AB BB (PDGF-AB BB), and vascular endothelial growth factor A (VEGF-A), as a subject having an FRI; and
administering to said subject identified having an FRI, an anti-infection therapy; or
II) identifying a subject having a spectroscopic profile of their plasma sample that exhibits a higher absorbance at about 1624-1625 cm⁻¹ and/or about 1188-1189 cm⁻¹ and/or lower absorbance at about 610-611 cm-1, 1592-1593 cm-1, 1648-1649 cm⁻¹, and/or 3288-3289 cm⁻¹ relative to a control spectroscopic profile; and
administering to said identified subject an FRI an anti-infection therapy; or
III) a) receiving an identification of the subject as having
i) a spectroscopic profile of a plasma sample associated with FRI or
ii) an increased relative expression of one or more proteins selected from a test profile comprising interleukin 6 (IL-6), platelet-derived growth factor AB BB (PDGF-AB BB), and vascular endothelial growth factor A (VEGF-A); and
b) administering to said subject identified as having an FRI, an anti-infection therapy, optionally wherein the anti-infection therapy is the administration of antibiotics.

18. The method of claim 16 wherein the subject is identified as having an FRI by
determining if a subject's plasma sample exhibits a biomarker associated with FRI, wherein the biomarker is
i) a spectroscopic profile of a plasma sample associated with FRI or
ii) an increased relative expression of one or more proteins selected from the group consisting of interleukin 6 (IL-6), platelet-derived growth factor AB BB (PDGF-AB BB), and vascular endothelial growth factor A (VEGF-A).

19. The method of claim 18 wherein the steps of identifying a spectroscopic profile of a plasma sample associated with FRI comprises:
obtaining a test spectroscopic profile of a plasma sample obtained from the subject; and
comparing the test spectroscopic profile to a control spectroscopic profile of a plasma sample,
wherein higher absorbance at about 1624-1625 cm⁻¹ and/or about 1188-1189 cm⁻¹ and/or lower absorbance at about 610-611 cm⁻¹, 1592-1593 cm⁻¹, 1648-1649 cm⁻¹, and/or 3288-3289 cm⁻¹ of the test spectroscopic profile when compared to the control spectroscopic profile indicates that the subject has an FRI.

20. The method of claim 18 wherein the steps of identifying elevated levels of one or more proteins selected from the group consisting of interleukin 6 (IL-6), platelet-derived growth factor AB BB (PDGF-AB BB), and vascular endothelial growth factor A (VEGF-A) comprises:
obtaining a test protein biomarker profile of a plasma sample obtained from said test subject; and
comparing the test protein biomarker profile to a control protein biomarker profile of a plasma sample,
wherein elevated expression of at least one of the proteins in the test protein biomarker profile relative to the control protein biomarker profile identifies a patient having an FRI.

21. The method of claim 20 wherein the steps of determining if the subject has elevated levels of said three or more proteins comprises detecting at least three of the following:
i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL in a plasma sample form said subject.

22. The method of claim 21, wherein said proteins are quantified using an antibody that specifically binds to the respective proteins.

24. A method of measuring a spectroscopic profile of a plasma sample wherein said method comprises
obtaining a test spectroscopic profile of a plasma sample obtained from a subject wherein absorbance is measured at
i) about 1624-1625 cm⁻¹;
ii) about 1188-1189 cm⁻¹;
iii) about 610-611 cm⁻¹;
iv) about 1592-1593 cm⁻¹;
v) about 1648-1649 cm⁻¹; and
vi) about 3288-3289 cm⁻¹.
